(19) United States
(12) Patent Application Publication
(10) Pub. No.: US 2017/0000380 A1
(43) Pub. Date: Jan. 5, 2017

(54) METHOD AND SYSTEM FOR ELECTROMAGNETIC TRACKING WITH MAGNETIC TRACKERS FOR RESPIRATORY MONITORING

(71) Applicant: KONINKLIJKE PHILIPS N.V., Eindhoven (NL)

(72) Inventor: Shlomo GOTMAN, HAIFA (IL)

(21) Appl. No.: 15/102,566

(22) PCT Filed: Dec. 4, 2014
(86) PCT No.: PCT/IB2014/066571

§ 371 Date: Jun. 8, 2016

Related U.S. Application Data
(60) Provisional application No. 61/915,163, filed on Dec. 12, 2013.

(51) Int. Cl. A61B 5/06; A61B 6/00; A61B 5/00; A61B 6/12; A61B 5/05; A61B 5/08

(52) U.S. Cl. CPC A61B 5/062; A61B 5/05; A61B 5/064; A61B 5/066; A61B 5/0816; A61B 5/6823; A61B 5/7289; A61B 6/12; A61B 6/541; A61B 2034/2051

(57) ABSTRACT

A method and system using electromagnetic tracking to monitor the respiration of a patient. The system includes trackers attached to the patient that emit and receive a magnetic field that changes as the patient breathes. The changing field received by the tracker can be associated with the breathing states of the patient and used to generate a respiratory signal. The respiratory signal may be used to indicate when to advance an intervention tool during an intervention procedure on the patient. The same electromagnetic system may also be used to track the position of the intervention tool, further assisting the intervention procedure.

The present application relates generally to a method and system for respiration-based guidance during interventional procedures. Interventional procedures can include interactive image-guided surgery and interactive surgical procedures, such as biopsies. It finds particular application with minimally invasive surgical procedures performed in conjunction with x-ray computed tomography (CT) imaging systems. These procedures involve the use of surgical tools for biopsy or brachytherapy needles or the like for tissue sampling or planning or placement of objects or instruments within the body of a subject, such as a patient. It is to be appreciated, however, that the invention is also applicable to a wide range of other imaging equipment and techniques, for example ultrasonic and magnetic resonance imaging devices, PET, SPECT, etc., and to a broad range of minimally invasive surgical procedures including many forms of surgery for placing objects or instruments at precise locations within a patient, such as interventional radiology procedures and others. A typical goal of interventional procedures, along with most procedures involving radiation, is to control the radiation delivery to minimize radiation exposure to the patient and the technician, such as a radiologist.

A CT scanner is commonly used for image guidance during interventional procedures. While it is possible to continuously watch the needle advancement using CT fluoroscopy, this method is seldom used due to much higher radiation dose for the patient, hand exposure to the primary beam for the radiologist, and inconvenience to manipulate the tool, such as, for example, a needle, inside a CT gantry bore. Therefore, a common practice is to use incremental tool advancement with periodic verification of the tool position by a single CT shot or scan.

One of the major challenges during such procedures is respiratory motion. Due to such motion, the position of the internal organs in the target area during tool manipulation can differ significantly from the position of the internal organs during the prior CT scan.

Respiratory monitoring has been used in the context of CT-guided procedures to select the optimal time for a CT scan, primarily to avoid motion artifacts (e.g., by using respiratory gating) or for post-procedure analysis of the recorded respiration wave (e.g., for radiation therapy planning).

One common practice used to address respiratory motion during an interventional procedure is to have the patient hold his breath at the same level during both the CT scan and afterwards, during the intervention tool insertion. However, various respiratory monitoring techniques used with breath-holding and other breath-control protocols may not accurately determine the respiratory states of the subject. In addition, the components of respiratory monitoring systems typically serve only one purpose—to monitor respiration. Intervention tool guidance systems are also typically stand-alone systems that only monitor the location and position of the intervention tool during the intervention procedure.

The proposed system and method allow a user, such as, for example, a technician, radiologist, or surgeon, to accurately monitor respiration using an electromagnetic tracking system and thereby limit manipulation of the interventional tool to times when the positions of the internal organs are close to their positions during a prior CT scan. The method is based on continuous respiratory monitoring of the patient, both during the scan and during tool advancement throughout the interventional procedure, with, in one embodiment, audio and/or visual notification of when to start and/or stop the tool manipulation, for example, needle advancement.

In one embodiment, an electromagnetic tracking system includes a first tracker attached to a subject, wherein the first tracker includes an emitter to emit a magnetic field, a second tracker attached to the subject, wherein the second tracker includes a first receiver to measure the magnetic field, and logic to determine a position of the second tracker relative to the first tracker based on the magnetic field measured by the first receiver and to generate a respiratory signal based on the position of the second tracker relative to the first tracker, wherein the position of the second tracker relative to the first tracker changes during a plurality of respiratory states of the subject.

Numerous advantages and benefits will become apparent to those of ordinary skill in the art upon reading the following detailed description of several embodiments. The invention may take form in various components and arrangements of components, and in various process operations and arrangements of process operations. The drawings are only for the purpose of illustrating many embodiments and are not to be construed as limiting the invention.

The descriptions of the invention do not limit the words used in the claims in any way or the scope of the claims or invention. The words used in the claims have all of their full ordinary meanings.

In the accompanying drawings, which are incorporated in and constitute a part of the specification, embodiments of the invention are illustrated, which, together with a general description of the invention given above, and the detailed description given below, serve to exemplify embodiments of this invention.

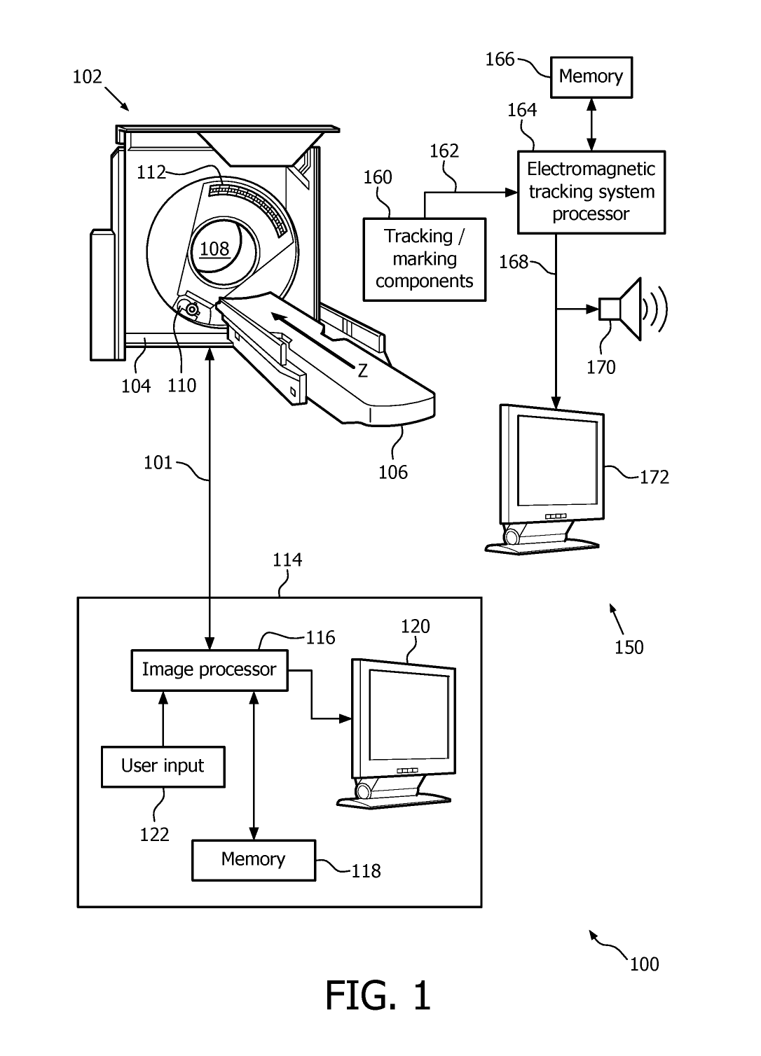

FIG. 1 illustrates an exemplary CT imaging system with an exemplary electromagnetic tracking system;

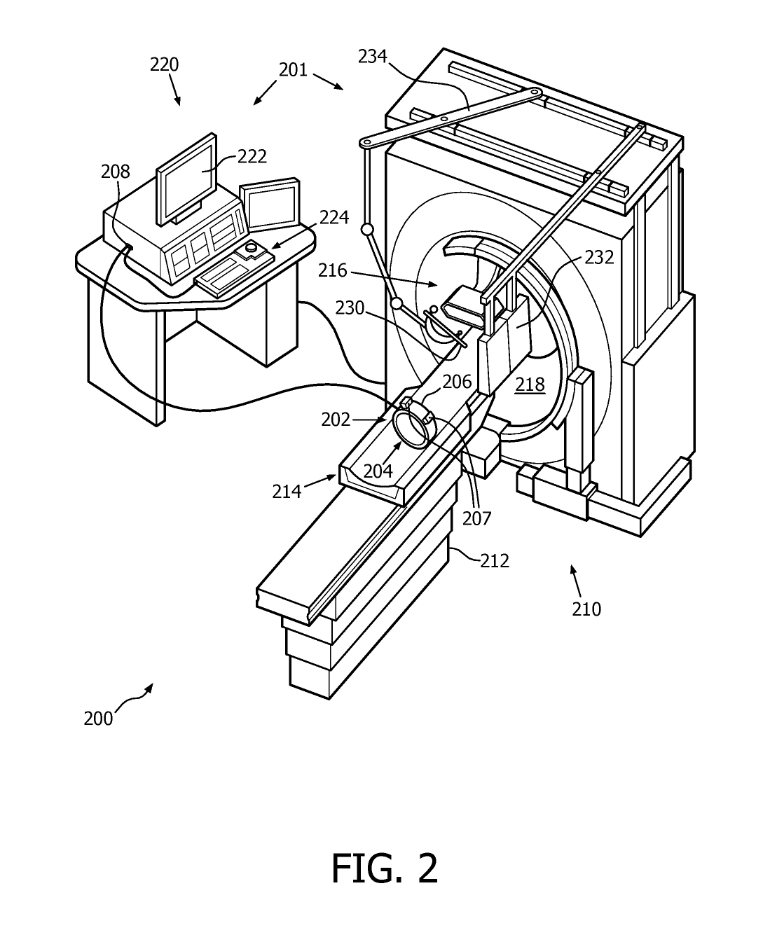

FIG. 2 illustrates an exemplary integrated apparatus with exemplary respiratory monitoring, imaging, and interventional procedure systems;

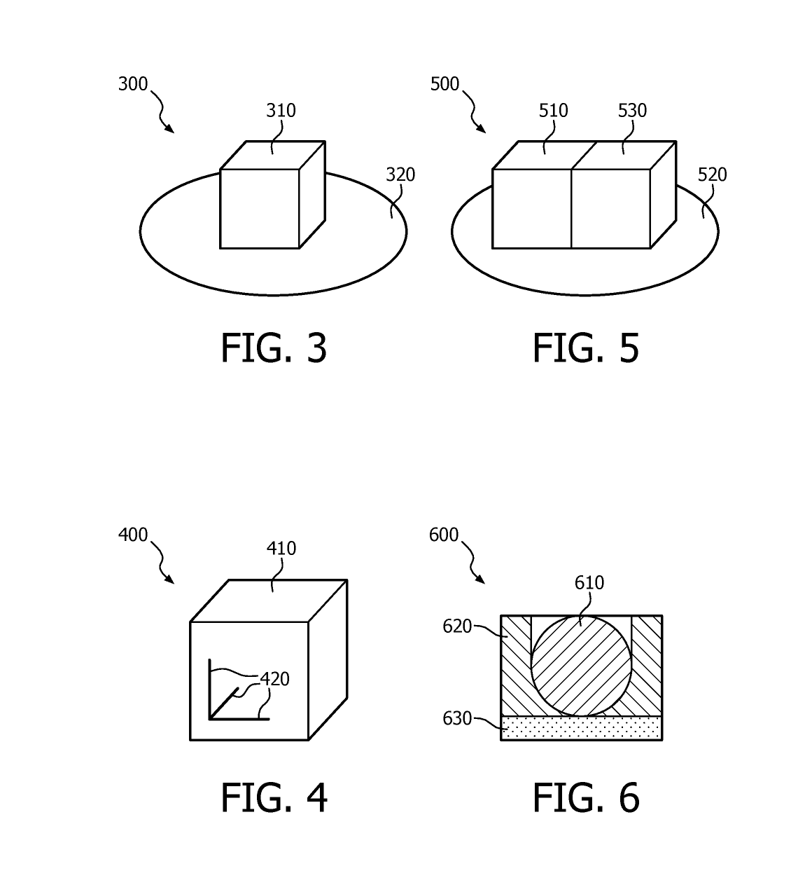

FIG. 3 is a drawing of an exemplary electromagnetic tracking assembly;

FIG. 4 is a drawing of another exemplary electromagnetic tracking assembly showing an exemplary three-coil configuration;

FIG. 5 is a drawing of an exemplary universal device;

FIG. 6 is a cross-sectional drawing of an exemplary multimodal fiducial marker;

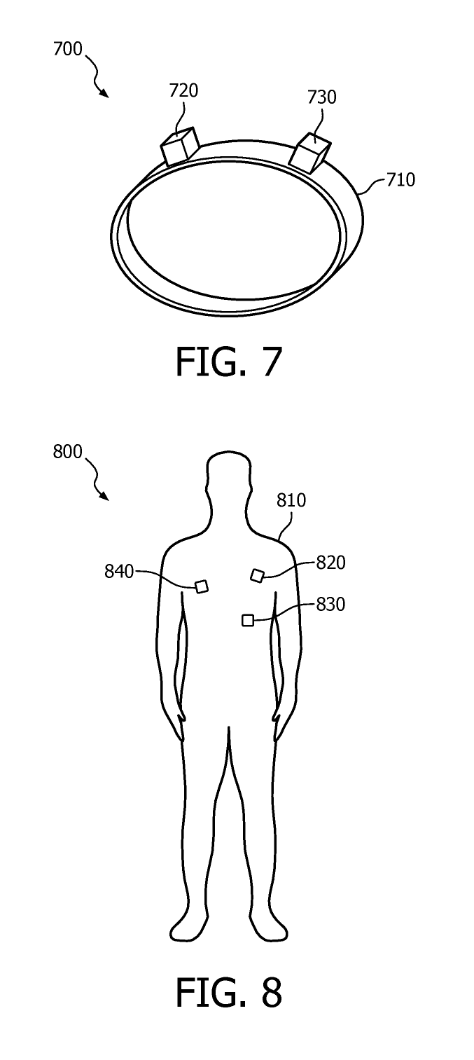

FIG. 7 is a drawing of components of an exemplary respiratory monitor utilizing an electromagnetic tracking system;

FIG. 8 is a drawing of components of another exemplary respiratory monitor on a patient using an electromagnetic tracking system;

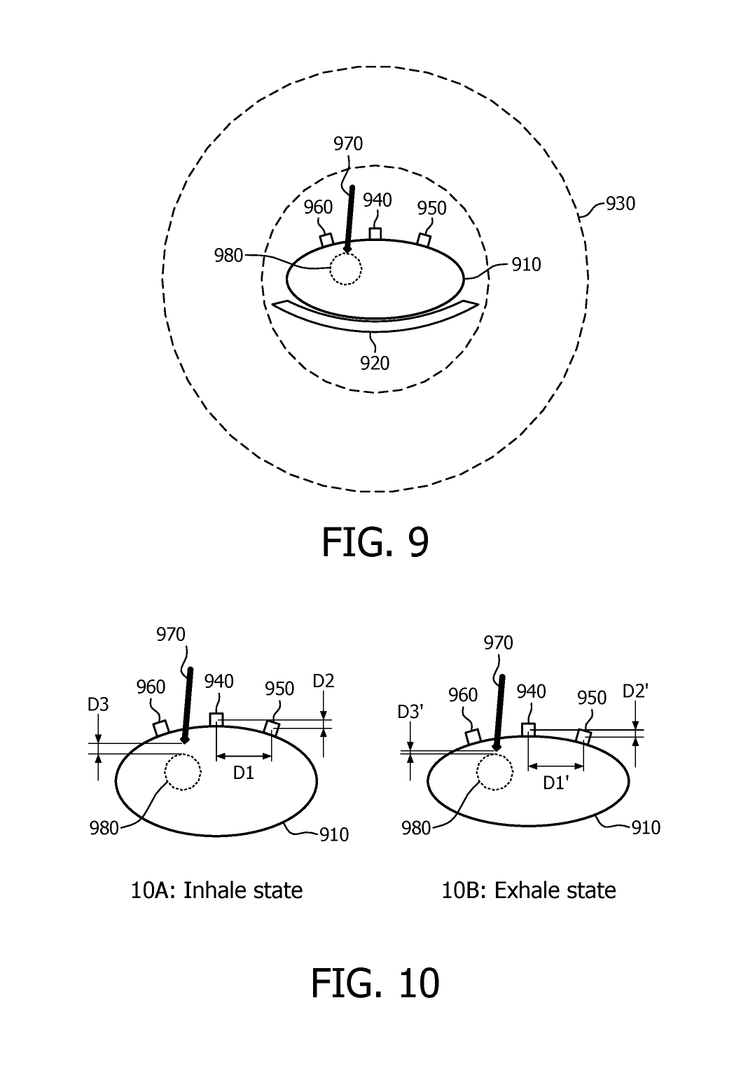

FIG. 9 is a cross-sectional drawing of a patient undergoing an interventional procedure with exemplary electromagnetic tracking devices;

FIG. 10 includes cross-sectional drawings of a patient undergoing an interventional procedure;

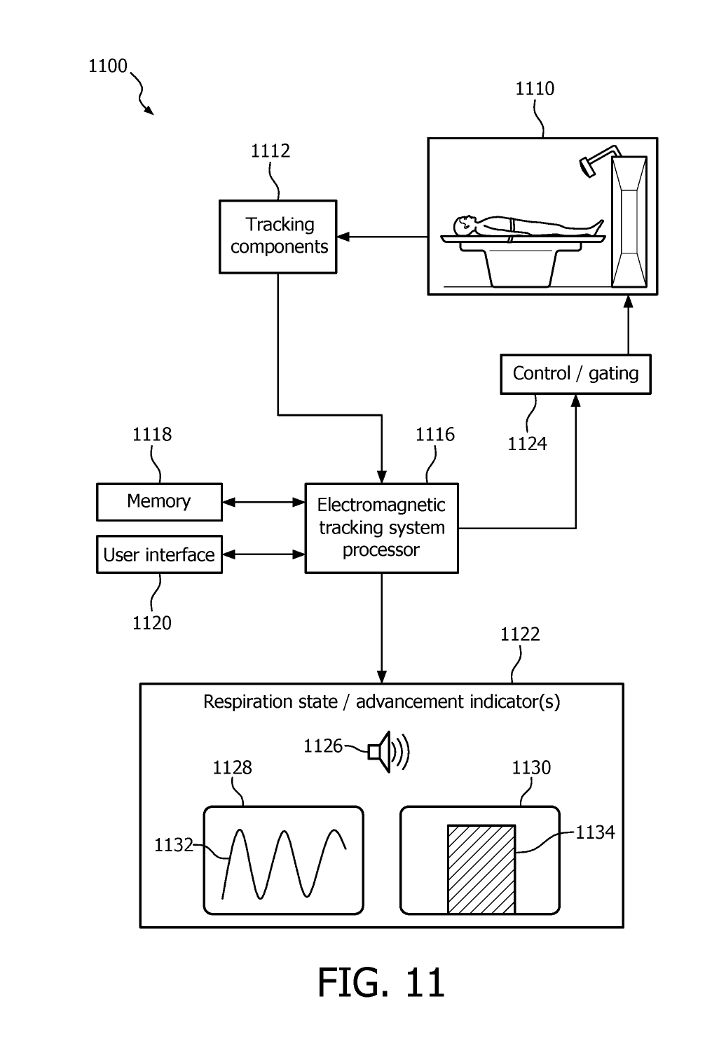

FIG. 11 is a schematic/block diagram representation of an exemplary system for respiratory monitoring and indicating intervention tool advancement timing during an intervention procedure;

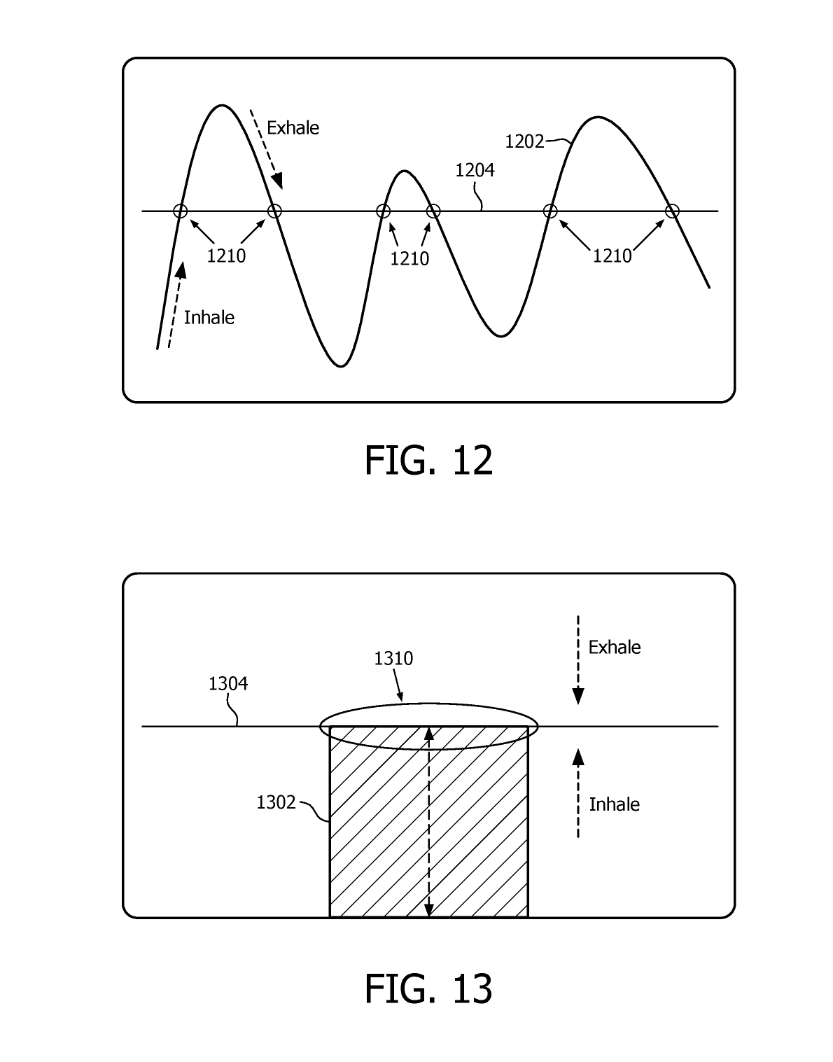

FIG. 12 shows an exemplary waveform representing a patient's respiration state during normal breathing;

FIG. 13 shows an exemplary bar graph having a height representative of an inhalation/exhalation level of a patient;

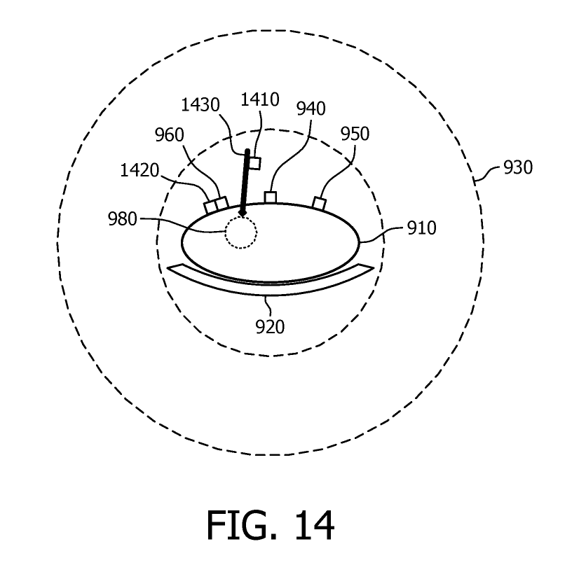

FIG. 14 is a cross-sectional drawing of a patient undergoing an interventional procedure with exemplary electromagnetic tracking devices to monitor the patient's respiration and the location and orientation of an intervention tool;

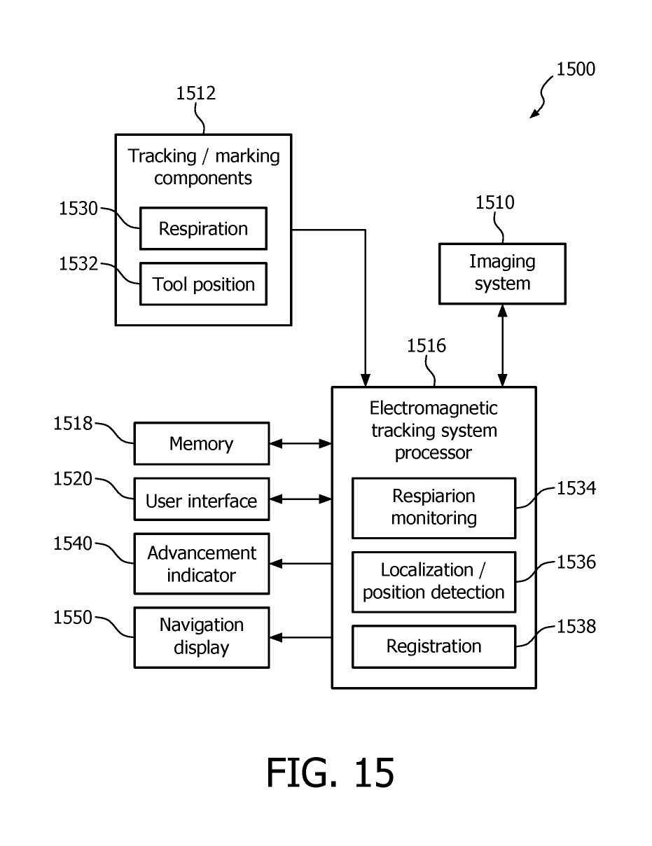

FIG. 15 is a block diagram representing an exemplary system 1500 for respiratory monitoring, intervention tool navigation, and indicating intervention tool advancement timing;

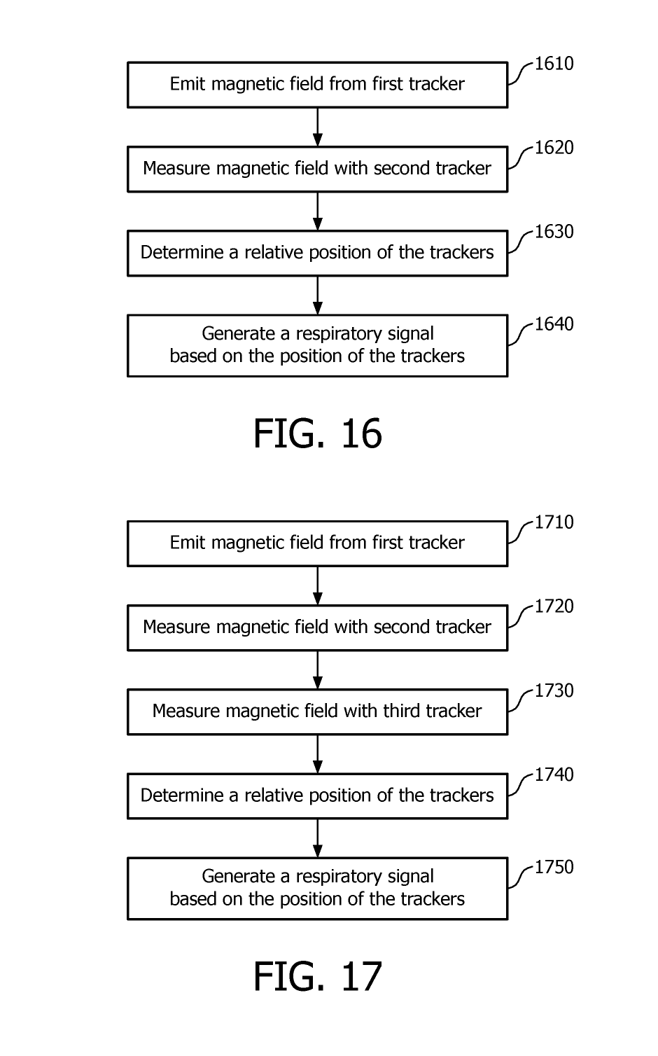

FIG. 16 is a flowchart of an exemplary method of generating a respiratory signal using an electromagnetic respiration monitor;

FIG. 17 is a flowchart of another exemplary method of generating a respiratory signal using an electromagnetic respiration monitor;

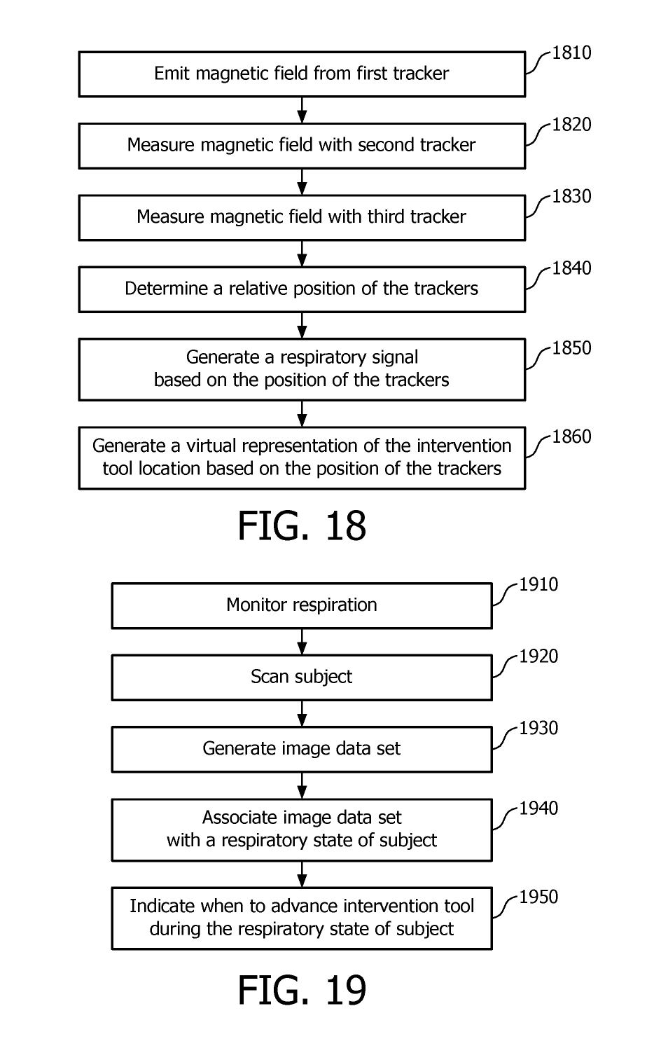

FIG. 18 is a flowchart of an exemplary method of generating a respiratory signal and a virtual representation of an intervention tool using an electromagnetic tracking system; and

FIG. 19 is a flowchart of an exemplary method of indicating when to advance an intervention tool during an intervention procedure.

In one embodiment, an exemplary CT imaging system 100 and an exemplary electromagnetic tracking system 150 are shown in FIG. 1. A CT imaging acquisition system 102 includes a gantry 104 and a table or other support 106 which may move along the z-axis. A patient or other subject to be imaged (not shown in FIG. 1) lies down on the table 106 and is moved to be disposed within an aperture or bore 108 in the gantry 104. Once the patient is in position, an x-ray source 110 and an x-ray detector 112 rotate together around the bore 108 to record CT imaging data. Other imaging system modalities may also be used in conjunction with the claimed invention, including, for example, cone beam CT, other x-ray based imaging, ultrasound imaging, magnetic resonance imaging (MRI), positron emission tomography (PET) imaging, and the like.

The CT imaging acquisition system 102 can then pass the CT imaging data on to a CT imaging processing and display system 114 through a communication link 101. Although the systems 102 and 114 are shown and described here as being separate systems for purposes of illustration, they may in other embodiments be part of a single system. The CT imaging data passes to an image processor 116 which can store the data in a memory 118. The image processor 116 electronically processes the data to perform an image reconstruction. The image processor 116 can show the resulting images on an associated display 120. A user input 122 such as a keyboard and/or mouse device may be provided for a user to control the processor 116.

An exemplary electromagnetic tracking system 150 is also shown in FIG. 1. The tracking system 150 includes tracking and/or marking components 160, such as trackers and/or markers that can monitor movement associated with a subject's respiration and register components with the subject's image data, as described in more detail below. The tracking and/or marking components 160 can provide one or more signals 162 to an electromagnetic tracking system processor 164, which can store data and other information in a memory 166. The electromagnetic tracking system processor 164 may include logic for determining relative movement and/or position between the tracking and/or marking components 160. The electromagnetic tracking system processor 164 may also produce an intervention tool advancement indicator signal 168, which can indicate when the patient's respiration state signifies that the patient's position is suitable for advancing an intervention tool (not shown). In another embodiment, the electromagnetic tracking system processor 164 can provide a signal to an intervention advancement processor, which can produce the intervention tool advancement indicator signal 168. In another embodiment, these processors may be combined. The advancement indicator signal 168 may be used to drive one or more indicating devices, such as, for example, an audible indicator, such as speaker 170, and/or a visual indicator, such as display 172.

Many of the aforementioned functions can be performed as software logic. “Logic,” as used herein, includes but is not limited to hardware, firmware, software and/or combinations of each to perform a function(s) or an action(s), and/or to cause a function or action from another component. For example, based on a desired application or needs, logic may include a software controlled microprocessor, discrete logic such as an application specific integrated circuit (ASIC), or other programmed logic device. Logic may also be fully embodied as software.

“Software,” as used herein, includes but is not limited to one or more computer readable and/or executable instructions that cause a computer, processor, or other electronic device to perform functions, actions, and/or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory such as memories 118 and 166, part of an operating system or other type of executable instructions. It will be appreciated by one of ordinary skill in the art that the form of software is dependent on, for example, requirements of a desired application, the environment it runs on, and/or the desires of a designer/programmer or the like.

The systems and methods described herein can be implemented on a variety of platforms including, for example, networked control systems and stand-alone control systems. Additionally, the logic shown and described herein preferably resides in or on a computer readable medium such as the memory 118 and/or 166. Examples of different computer readable media include Flash Memory, Read-Only Memory (ROM), Random-Access Memory (RAM), programmable read-only memory (PROM), electrically programmable read-only memory (EPROM), electrically erasable programmable read-only memory (EEPROM), magnetic disk or tape, optically readable mediums including CD-ROM and DVD-ROM, and others. Still further, the processes and logic described herein can be merged into one large process flow or divided into many sub-process flows. The order in which the process flows herein have been described is not critical and can be rearranged while still accomplishing the same results. Indeed, the process flows described herein may be rearranged, consolidated, and/or re-organized in their implementation as warranted or desired.

The exemplary electromagnetic tracking system 150 may be a stand-alone system or in other embodiments may be fully or partially integrated with the exemplary CT imaging system 100 to form a combined system.

Referring now to FIG. 2, another embodiment is shown with an exemplary integrated apparatus 200, which includes an exemplary imaging system 201 and an exemplary electromagnetic tracking system 202. The integrated apparatus 200 is particularly well suited for planning and executing minimally invasive interventional procedures, such as, for example, biopsies, in-vivo placement of instruments and/or objects within a patient, etc., as described above.

The electromagnetic tracking system 202 includes tracking and/or marking components 204, which can facilitate respiratory monitoring, image registration, and intervention tool navigation, as discussed in more detail below. In one embodiment, the tracking and/or marking components 204 may include a belt 206 adapted for attachment around the abdomen or chest of a patient and two or more electromagnetic tracking devices 207. In this embodiment, the relative movement of the electromagnetic tracking devices 207 can be used for generating a signal corresponding to the displacement of a patient's abdomen during respiration. The tracking and/or marking components 204 are connected to the imaging system 201 at a suitable electronic connection point 208.

With continued reference to FIG. 2, the imaging system 201 includes a volumetric diagnostic CT imaging apparatus/scanner 210 as shown. The CT imaging apparatus 210 is disposed in axial alignment with a patient table 212 and support 214 such that a patient or subject on the support 214 surface can be moved into and through a bore 216 of the CT scanner 210. The CT scanner 210 includes an x-ray tube mounted for rotation about a pre-selected plane. The x-ray tube can project a fan shaped beam of radiation through a ring 218 of radiation translucent material, through the patient support 214, through a region of interest or target area of the patient, and to a ring or arc of radiation detectors disposed opposite the x-ray tube. As the x-ray tube rotates within the plane, a series of data lines are generated, which data lines are reconstructed into at least a slice image using well known techniques by a reconstruction processor included in a control console 220 operatively connected with the CT scanner 210.

As is well known in the art, in some embodiments, the patient support 214 can move longitudinally along the z-axis as the x-ray tube is rotating around the subject such that a selected volume of the patient is scanned along a spiral path or a series of slices. The position of the x-ray tube is monitored by a rotational position encoder and the longitudinal position of the patient support is monitored by similar position encoders disposed within the table 212. In other embodiments, volumetric data may be obtained without longitudinal movement. The reconstruction processor can reconstruct a volumetric image representation from the generated data lines. The control console 220 includes one or more human readable display devices, which can be in the form of an operator monitor or display 222 and at least one operator input device 224, such as, for example, a keyboard, track ball, mouse, or the like.

With continued reference to FIG. 2, an exemplary intervention tool 230 is shown above the patient support 214. An exemplary intervention tool advancement indicator device 232 is shown opposite the intervention tool 230. The intervention tool advancement indicator device 232 may include, for example, an audible indicator, such as speaker 170, and/or a visual indicator, such as display 172, as shown in FIG. 1. The intervention tool 230 and intervention tool advancement indicator device 232 are shown supported from overhead on a track or by other means 234 atop the CT scanner 210. In this embodiment, the intervention tool 230 and intervention tool advancement indicator device 232 are shown in exemplary positions that may be convenient for a user performing an exemplary interventional procedure on the subject. However, the positioning of any of the devices shown in this embodiment may be arranged or supported differently in other embodiments. The intervention tool advancement indicator device 232 can be oriented or moved into selected positions on the support system 234 for easy viewing and/or hearing by a user.

As shown in FIG. 2, the exemplary control console 220 may include both an image processor, an electromagnetic tracking system processor, and an intervention advancement processor (not shown), such as, for example, the image processor 116 and the electromagnetic tracking system processor 164 of FIG. 1. In various embodiments, these processors may be physically and/or electronically integrated within the control console 220 and/or each other. In another embodiment, the electromagnetic tracking system processor 164 may be included in a device with the intervention tool advancement indicator device 232 and/or the intervention advancement processor.

FIG. 3 is a drawing of an exemplary electromagnetic tracking assembly 300. The assembly 300 includes an exemplary electromagnetic tracking device or tracker 310 and an exemplary base 320 for attachment to the subject or patient, via, for example, an adhesive layer. The electromagnetic tracking device 310 may be one of the tracking and/or marking components 204 as mentioned above in relation to FIG. 2, and in particular, one of the electromagnetic tracking devices 207. The electromagnetic tracking device 310 can be used as part of spatial tracking or localizing system based on known electromagnetic technology. In particular, the electromagnetic tracking device 310 includes an emitter that emits a magnetic field generated by one or more coils or a receiver that measures the magnetic field around itself when positioned in the emitted magnetic field using one or more coils. An electromagnetic tracking system, such as, for example, system 202 shown in FIG. 2, requires at least one electromagnetic tracking device 310 with an emitter and at least one electromagnetic tracking device 310 with a receiver.

A processor, such as, for example, the electromagnetic tracking system processor 164 of FIG. 1, can utilize signals from the electromagnetic tracking devices 310, which are indicative of the magnetic field emitted by an emitter and measured by a receiver, to determine the relative position and/or orientation of the electromagnetic tracking devices 310. For example, electromagnetic tracking devices 310 with single coil emitter and receiver designs may allow for the determination of the relative distance between the electromagnetic tracking devices 310, whereas electromagnetic tracking devices 310 with multiple coils may allow for the determination of the relative distance and orientation of the electromagnetic tracking devices 310. Electromagnetic localizing systems are ergonomic and easy to use because direct line-of-sight visualization is not required, allowing obstacles to be positioned between the emitter and the receiver, including, for example, a hand, a surgical drape, an intervention tool, etc., without impacting the accuracy of the spatial measurements. Although the electromagnetic tracking devices 207, 310 shown in FIGS. 2 and 3 are drawn as relatively large cubes, the electromagnetic tracking devices 207, 310 and any others mentioned herein may be any size and/or shape suitable for any particular application of an electromagnetic tracking system.

FIG. 4 is a drawing of an exemplary electromagnetic tracking device 400 including an outer covering 410 and three coils 420 imbedded inside the device 400. In this embodiment, the three coils 420 are all oriented orthogonally to each other, forming a three-axis spatial reference grid or coordinate system. Two-coil designs may also be suitable for certain applications. Although the three coils 420 are shown with their ends meeting at a common vertex, this need not be the case. In other embodiments, coils may be separated and oriented in non-orthogonal configurations.

FIG. 5 is a drawing of an exemplary universal device 500 that includes an exemplary electromagnetic tracking device 510, an exemplary base 520 for attachment to the subject or patient, and an exemplary fiducial marker 530. The electromagnetic tracking device 510 may be one of the electromagnetic tracking devices 310, 410 mentioned above. Although shown together in universal device 500, in other embodiments the electromagnetic tracking device 510 and fiducial marker 530 may be used as separate components, for example, as part of the tracking and/or marking components 160 shown in FIG. 1. In other embodiments, the electromagnetic tracking device 510 and fiducial marker 530 may be integrated into one housing or covering.

The fiducial marker 530 allows for image registration. In particular, the fiducial marker 530 is made of a material that can be detected during the scan of the patient by, for example, the CT scanner 210 shown in FIG. 2. However, in addition to CT, imaging modalities may include fluoroscopy, positron emission tomography (“PET”), micro PET, single photon emission computed tomography (“SPECT”), micro SPECT, magnetic resonance (“MR”), ultrasound, and others. Although shown in FIG. 5 as a cube, in other embodiments, the fiducial marker 530 and any others mentioned herein may be any size, shape, and material suitable for detection and registration of the image data from the scan associated with the interventional procedure, including multimodal fiducial markers for registration of different types of imaging data.

By including the fiducial marker 530 in the view of a scan of a patient, the physical location of the fiducial marker 530 relative to the area of interest in the patient, such as, for example, the target area of an interventional procedure, is known. The physical location of the fiducial marker 530 relative to the electromagnetic tracking device 510 is also known. By combining this information, the acquired images can be aligned in a common spatial reference grid or coordinate system for navigation of an intervention tool (also outfitted with a tracking device) to the target area, as discussed in more detail below.

FIG. 6 is a cross-sectional drawing of an exemplary multimodal fiducial marker 600 that may be used as the fiducial marker 530. More specifically, a first portion 610 of the multimodal fiducial marker 600 may be a radiopaque material. A radiopaque material is a material which is opaque to x-ray radiation, so it is visible in x-ray photographs and under fluoroscopy. Materials which are dense enough to be opaque to x-ray radiation are also typically visible in an ultrasound scan. Thus the first portion 610 will be visible to CT, fluoroscopy, and other x-ray based imaging systems, as well as ultrasound imaging systems. The first portion 610 of the exemplary fiducial marker 600 may be advantageously shaped in the form of a sphere. Such a spherical geometry allows the first portion 610 to be consistently identified from all angles and allows for accurate localization of the center of the fiducial marker 600. The spherical shape of the first portion 610 also allows the fiducial marker 600 to be easily detectable such that the image registration process may be automated. However, other shapes and geometries known in the art may be used. The second portion 620 of the multimodal fiducial marker 600 may be radioactive. For example, the second portion 620 may be comprised of a porous material which absorbs a radioactive material. Once the porous material is at least partially saturated with the radioactive material, the second portion 620 is activated. The resulting radioactivity of the second portion 620 will be visible to PET, micro PET, SPECT, and micro SPECT imaging systems. The fiducial marker 600 also includes an attachment means for attaching the fiducial marker 600 to a base or the imaged subject, or an apparatus on or in which the imaged subject is disposed.

FIG. 7 is a drawing of components of an exemplary respiratory monitor 700 utilizing an electromagnetic tracking system, similar to the tracking and/or marking components 204, shown in FIG. 2. In this embodiment, the respiratory monitor 700 includes a belt 710 adapted for attachment around the abdomen or chest of a patient and two electromagnetic tracking devices 720, 730. FIG. 8 is a drawing of components of another exemplary respiratory monitor 800 on a patient 810 using an electromagnetic tracking system. In this embodiment, the respiratory monitor 800 includes three electromagnetic tracking devices 820, 830, 840 attached directly to the patient 810. In other embodiments, any of the electromagnetic tracking devices 720, 730, 820, 830, 840 shown in FIGS. 7 and 8 can be replaced with or enhanced with one or more additional electromagnetic tracking devices, fiducial markers, and/or universal devices for use in other applications.

FIG. 9 is a cross-sectional drawing of a patient 910 undergoing an interventional procedure with exemplary electromagnetic tracking devices 940, 950, 960 used to monitor the patient's respiration. The patient 910 is shown on a support 920 in front of a bore of an exemplary CT scanner 930. An exemplary intervention tool 970 is also shown advancing towards a target area 980 within the patient 910. As described above, the electromagnetic tracking devices 940, 950, 960 are part of an electromagnetic tracking system used to monitor the respiration of the patient 910 during scans and the intervention procedure.

Generally, the electromagnetic tracking system tracks the respiration of the patient using the electromagnetic tracking devices 940, 950, 960 and can generate a signal or other representation of the patient's respiration state. The signal may be used to indicate to the user when to advance the intervention tool 970. In particular, the intent is to advance the intervention tool 970 when the patient's respiration state is at the same state as when the patient 910 was scanned to produce an image data set. As discussed above, the patient's internal organs and chest cavity may move during respiration, but this movement can be correlated to the patient's respiration states. It is beneficial and preferable to advance the intervention tool 970 when the user knows where the patient's internal organs and chest cavity are positioned. Therefore, knowing the respiration state of the patient 910 during the scan, which is the source of the image data that the user uses to guide the intervention tool 970 during intervention tool advancement, allows the system to indicate to the user when the patient's internal organs and chest cavity will be in the same position again, i.e., when the patient 910 is in the same respiration state. In one embodiment, and as discussed in more detail below, the patient 910 may be asked to hold his breath when the system indicates that the patient's respiration state is the same state as when the scan was taken, thereby allowing the user to advance the intervention tool 970 while the patient is in the same position as when the scan was taken. In another embodiment, and also discussed in more detail below, the patient 910 may breathe normally, such that when the system indicates that the patient's respiration state is the same or is about to be in the same as when the scan was taken, the user can advance the intervention tool 970 a relatively small amount while the patient 910 is in the same position as when the scan was taken.

An electromagnetic tracking system processor can include logic to correlate the relative positions of the electromagnetic tracking devices 940, 950, 960 to a patient's respiration state and generate a respiratory signal or respiration representation. FIG. 10 includes cross-sectional drawings of the patient 910 undergoing the interventional procedure. The patient's respiration is monitored with electromagnetic tracking devices 940, 950, 960, shown in an inhale state (FIG. 10A, where the cross-section of the patient's chest cavity is more rounded) and in an exhale state (FIG. 10B, where the cross-section of the patient's chest cavity is flatter). In this simplified example, the orientations and relative distances between electromagnetic tracking devices 940 and 950 are shown in two dimensions during the exemplary inhale and exhale states. As shown in FIG. 10A, D1 and D2 are the relative distances between devices 940 and 950 in two dimensions during an inhale state. Also during the inhale state, D3 is the distance between the tip of the intervention tool 970 and the target area 980. As shown in FIG. 10B, D1′ and D2′ are the relative distances between devices 940 and 950 during an exhale state. Also during the exhale state, D3′ is the distance between the tip of the intervention tool 970 and the target area 980. As shown in these drawings, the relative position of devices 940 and 950 changes during the exemplary inhale and states of the patient 910. In particular, D1 is less than D1′ and D2 is greater than D2′. These changes in relative position can be tracked and directly correlated to the respiration states of the patient 910. The significance of these changes is depicted by the change in the distance between the tip of the intervention tool 970 and the target area 980 during the inhale and exhale states. In particular, D3 is significantly greater than D3′, meaning that as the patient 910 exhales, the tip of the intervention tool 970 is drawn closer to the target area 980, without any advancement of the intervention tool 970 by the user. In other embodiments, the relative positions of devices 940 and 950 may be tracked in three dimensions and device 960 and possibly other devices may also be used to monitor respiration. In other embodiments, spatial orientations may also be tracked in addition to or instead of relative positions, including the use of angular measures and relationships. References to “position” in this application can include both position and orientation. Different intervention procedures may require higher or lower degrees of accuracy, may involve more or less patient movement during respiration, etc., requiring different configurations of the electromagnetic tracking system and/or tracking algorithms.

FIG. 11 is a schematic/block diagram representation of an exemplary system 1100 for respiratory monitoring and indicating intervention tool advancement timing during an intervention procedure. System 1100 includes an exemplary scanning system 1110, such as, for example, a CT scanner. The system 1100 further includes exemplary tracking components 1112, such as, for example, electromagnetic tracking devices, a belt, etc., as mentioned above. In other embodiments, markers, such as, for example, fiducial markers, may be incorporated for image registration.

As illustrated, an electromagnetic tracking system processor 1116 receives one or more signals from the tracking components 1112 and is operatively connected with several components of the system 1100, including a memory 1118, a user interface 1120, one or more intervention tool advancement indicators 1122, and a scanning system controller/gating device 1124. The memory 1118 may be used to store various software, logic, and/or parameters utilized by the electromagnetic tracking system processor 1116, including, for example, measured values and parameters associated with the components 1112 and the respiration states of the subject, associated intervention tool triggering/threshold values and/or levels, algorithms for determining triggering/threshold points and/or levels, user selected values, image data, etc., as discussed in more detail below. The user interface 1120 can include user input and display devices and may be integrated as part of control console 220 of FIG. 2.

The exemplary intervention tool advancement indicators 1122 may include one or more audible indicators, such as speaker 1126, and/or one or more visual indicators, such as displays 1128, 1130. The speaker 1126 can produce a continuous sound, instances of the same sound, or instances of different sounds to indicate when to start and stop intervention tool advancement. For example, a continuous beep or “on” and “off” beeps may be used for indicating when to advance the intervention tool.

The displays 1128, 1130 can include visual indicia of the patient's breathing and/or indicate when to advance the intervention tool. In one embodiment, a display 1128, 1130 may simply display a visual cue, such as, for example, a word or color (e.g., red and/or yellow followed by green) to indicate when to advance the intervention tool. In other embodiments, as shown in FIG. 11, the displays 1128, 1130 can depict a representation of the patient's breathing. Exemplary display 1128 shows a waveform 1132 having an amplitude representing the respiration of a patient. Exemplary display 1130 shows a bar graph 1134 having a height representative of an inhalation/exhalation level of the patient on a scale of percentage of vital capacity (% VC). It is to be appreciated that although a waveform and bar graph are illustrated, other forms of patient breathing images can be used as well such as, for example, a graduated cylinder, a progress bar, an animated diaphragm, and the like.

In addition to driving intervention tool advancement indicators 1122, the electromagnetic tracking system processor 1116 can be used to provide a signal to control the scanning system 1110. For example, control/gating device 1124 may be used to control or provide a signal to the scanning system 1110 to scan the patient at a particular respiration state, for example, at the same respiration state during which the user was advancing the intervention tool. This may be helpful during confirmation scans. In one embodiment, the control/gating device 1124 may be integrated as part of control console 220 of FIG. 2.

An exemplary intervention procedure includes scanning stages and intervention tool advancement stages. During the scanning stage, a scanner scans a patient. An electromagnetic tracking system may be used to monitor the patient's breathing state during the scan, producing one or more signals indicative of the patient's breathing states. The patient may be asked to breathe normally or may be asked to hold his breath during scanning. The scanner produces an image data set, such as, for example, a volumetric data set, suitable to assist and guide the user during the subsequent intervention procedure. Exemplary image processing devices may be as described above in FIGS. 1 and 2. An intervention tool may be scanned with the target area of the patient, although this need not be the case in all embodiments. In some embodiments, it may be helpful to have the intervention tool scanned with the patient to establish a reference starting position of the intervention tool on the scan and in the image data set. During the scanning stage, one or more scans may be taken of the patient. For each scan, an electromagnetic tracking system processor reads the respiration signal(s) from the tracking components (e.g., electromagnetic tracking devices) and associates the signal readings with the respective image data set. The respiration signal(s) is also associated with a particular respiration state of the patient. All of this data may be saved in memory.

An intervention tool advancement stage follows the scanning stage. During the tool advancement stage, the patient is removed from the scanner, allowing for intervention tool advancement without additional exposure to radiation from the scanner. The electromagnetic tracking system continues to monitor the patient's breathing states and produces a respiration signal indicative of the patient's breathing states. The patient may be asked to breathe normally or may be asked to hold his breath during intervention tool advancement. The electromagnetic tracking system can drive an intervention tool advancement indicator that indicates when the patient's respiration state is at the same state as it was during the scan that created the image data set being used by the user to guide advancement of the intervention tool.

In this manner, the user is alerted to when the patient's internal organs and chest cavity are in the proper position to advance the intervention tool, by tracking the patient's respiration. In particular, the electromagnetic tracking system processor can monitor the patient's respiration state for certain thresholds/triggering points to drive the intervention tool advancement indicator, which indicates to the user a suitable time to advance the intervention tool. In particular, when the electromagnetic tracking system indicates that the patient's respiration state is the same as during the scan, via the intervention tool advancement indicator, an indication is provided to the user that it is a suitable time to advance the intervention tool, because the user knows that the patient's target area should be in a position matching the image data set.

FIGS. 12 and 13 show exemplary graphical representations of a patient's respiration states used for triggering an intervention tool advancement indicator. The representations shown in FIGS. 12 and 13 may be representative of the tracking and triggering algorithms utilized by an electromagnetic tracking system processor and/or an intervention advancement processor, which in some embodiments may also be displayed on a display associated with an intervention tool advancement indicator (e.g., display 172 in FIG. 1, displays 222, 232 in FIG. 2, and displays 1132, 1134 in FIG. 11).

FIG. 12 shows an exemplary waveform 1202 representing a patient's respiration state during normal breathing, but with variations in time and lung capacity fill levels from one breath to another. In this embodiment, waveform 1202 can be generated by the exemplary electromagnetic tracking system processor mentioned above. Waveform 1202 rises and falls over time while the patient inhales and exhales, respectively. The peaks and valleys shown in FIG. 12 may correspond to the inhale and exhale states shown in FIG. 10, respectively. Target line 1204 represents the respiration state corresponding to an associated image data set for use during the intervention procedure and from an earlier scan. Each time the patient's real-time respiration state 1202 is at (i.e., crosses) line 1204, the patient's respiration state (and the position of the patient's internal organs and chest cavity) corresponds to the respiration state (and position) associated with the image data set. In this embodiment, the processor uses these crossing points as triggering points 1210 to drive an intervention tool advancement indicator, which indicates to the user a suitable time to advance an intervention tool.

FIG. 13 shows an exemplary bar graph 1302 having a height representative of an inhalation/exhalation level of the patient on a scale of percentage of vital capacity (% VC). In this embodiment, bar graph 1302 rises and falls over time while the patient inhales and exhales, respectively. Target line 1304 represents the respiration state corresponding to an associated image data set for use during the intervention procedure and from an earlier scan. Each time the patient's real-time respiration state 1302 is at line 1304, the patient's respiration state (and the position of the patient's internal organs and chest cavity) corresponds to the respiration state (and position) associated with the image data set. In this embodiment, the patient may be asked to hold his breath when the graph is at line 1304, during which time the user can advance the intervention tool.

Various parameters and algorithms associated with the intervention tool advancement indicator, such as, start time, stop time, length of time to drive the indicator, responses to intervention, patient, and/or outside factors, etc., may be determined as part of a planning phase to develop an intervention procedure plan, which typically precedes the intervention procedure. In some embodiments, the intervention procedure plan may be modified during the intervention procedure, based on various factors, including, for example, a confirmation scan.

In addition to or instead of driving an intervention tool advancement indicator, the electromagnetic tracking system may be used to track the position and/or orientation of an intervention tool. FIG. 14 is a cross-sectional drawing of patient 910 undergoing an interventional procedure with exemplary electromagnetic tracking devices 940, 950, 960 used to monitor the patient's respiration, exemplary electromagnetic tracking devices 960, 1410 used to monitor the location and orientation of intervention tool 1430, and fiducial marker 1420 to register the electromagnetic tracking system with image data. In this embodiment, electromagnetic tracking device 960 includes an emitter that is used for tracking electromagnetic tracking devices 940, 950, 1410, which each include a receiver. In this manner, the same electromagnetic tracking system may be used to monitor the patient's respiration states, drive an intervention tool indicator, and/or track the position and orientation of an intervention tool 1430. In one embodiment, electromagnetic tracking device 960 and fiducial marker 1420 may be integrated into one universal device.

By including the fiducial marker 1420 in the view of a scan of the patient 910, the location of the fiducial marker 1420 relative to the target area 980 is known. The location of the fiducial marker 1420 relative to the electromagnetic tracking device 960 is also known. By combining this information, the acquired images can be aligned with the tracking components in a common spatial reference grid or coordinate system (i.e., localized space) for navigation of the intervention tool 1430, outfitted with electromagnetic tracking device 1410, to the target area 980. In sum, the fiducial marker 1420 is detected in the image data from the scanning system and used to register images of the patient 910 with components involved with the real-time interventional procedure. Associating the electromagnetic tracking device 960 with the fiducial marker 1420 enables defining a common referential system being utilized by both the imaging and localizing systems, so that data of the imaging and localizing systems can be registered together.

The spatial position and orientation of the intervention tool 1430 may be determined using electromagnetic tracking device 1410 in the same manner as described above in relation to the electromagnetic tracking devices 940, 950, 960 used for respiratory monitoring. In other embodiments, more than one electromagnetic tracking device may be associated with the intervention tool 1430. In some embodiments, the electromagnetic tracking device 1410 will be required to be a sophisticated three-coil receiver to accurately determine the spatial position and orientation of the whole intervention tool 1430 (instead of just the position of the electromagnetic tracking device 1410) in the localization system, for example, in applications where the path of the intervention tool 1430 advancement is important. Although the electromagnetic tracking device 1410 is shown at the end of the intervention tool 1430 away from the advancing tip in FIG. 14, the electromagnetic tracking device 1410 may be located at any location on the intervention tool 1430, including at the advancing tip. In embodiments where the electromagnetic tracking device 1410 is not located at the advancing tip of the intervention tool 1430, it is known in the art how to track the tip by knowing the relationship between the electromagnetic tracking device 1410 and the tip. To maintain suitable accuracy, in some embodiments, the offset distance between the electromagnetic tracking device 1410 and the tip of the intervention tool 1430 will be minimized.

In this manner, the position of the intervention tool 1430 can be correlated to the image data of the patient 910, allowing the system to virtually display the intervention tool 1430 in the images of the patient 910. The registration and tracking systems enable constructing and displaying a navigation image, wherein a virtual representation of the spatial position of the intervention tool 1430 is displayed on image data from the imaging system using the localization system defined by the electromagnetic tracking devices, including device 1410.

FIG. 15 is a block diagram representing an exemplary system 1500 for respiratory monitoring, intervention tool navigation, and indicating intervention tool advancement timing. System 1500 includes an exemplary imaging system 1510, such as, for example, a CT scanner. The system 1500 further includes exemplary tracking and/or marking components 1512, such as, for example, electromagnetic tracking devices and fiducial markers, to support respiration 1530 and intervention tool position 1532 tracking and registration functions.

As illustrated, an electromagnetic tracking system processor 1516 receives signals from the tracking components 1512 (e.g., electromagnetic tracking devices), indicative of respiration 1530 and tool position 1532, and image data from imaging system 1510. The processor 1516 may be operatively connected with several other components of the system 1500, including a memory 1518, a user interface 1520, one or more intervention tool advancement indicators 1540, and a navigation display 1550. The memory 1518 may be used to store various software, logic, values, and/or parameters utilized by the electromagnetic tracking system processor 1516, including, for example, measured values and parameters associated with the components 1512, the respiration states of the subject, the positions and orientations of the components 1512 and an intervention tool, associated intervention tool triggering/threshold values and/or levels, algorithms for determining triggering/threshold points and/or levels, user selected values, image data, registration data, tool dimensions, etc. The user interface 1520 can include user input and display devices and may be integrated as part of control console 220 of FIG. 2.

The electromagnetic tracking system processor 1516 can use the data and signals described above for the following functions: respiration monitoring 1534; localization and position/orientation detection 1536; and registration 1538. The electromagnetic tracking system processor 1516 can execute logic to perform the various calculations and determinations associated with each of these functions, as described above. These functions can be used by the electromagnetic tracking system processor 1516 to drive an intervention tool advancement indicator 1540 and intervention tool navigation display 1550. The navigation display 1550 can display the image data of a patient along with a virtual representation of the intervention tool in real time, including during the intervention procedure. The navigation display 1550 can be used by the user to guide the advancement of the intervention tool. The navigation display 1550 may be a separate display or may be incorporated with any of the displays mentioned above (e.g., display 172 in FIG. 1, displays 222, 232 in FIG. 2, and displays 1132, 1134 in FIG. 11). In one embodiment, the advancement indicator 1540 is incorporated with the navigation display 1550.

FIGS. 16-19 describe exemplary methods associated with utilization of electromagnetic tracking systems, including, for example, those mentioned above. Further embodiments of similar methods may include other additional steps, or omit one or more of the steps in the illustrated methods. Also, the order in which the process flows herein have been described may be rearranged while still accomplishing the same results. Thus the process flows described herein may be added to, rearranged, consolidated, and/or re-organized in their implementation as warranted or desired.

FIG. 16 is a flowchart of an exemplary method of generating a respiratory signal using an electromagnetic respiration monitor, such as those mentioned above. At step 1610, a first tracker attached to a subject emits a magnetic field. Next, at step 1620, a second tracker attached to the subject measures the magnetic field emitted by the first tracker. At step 1630, the relative position of the first and second trackers is determined based on the magnetic field measured by the second tracker. The changing field received by the tracker can be associated with the breathing states of the subject and used to generate a respiratory signal. At step 1640, the respiratory signal is generated based on the position of the second tracker relative to the first tracker.

FIG. 17 is a flowchart of another exemplary method of generating a respiratory signal using an electromagnetic respiration monitor, such as those mentioned above. Steps 1710-1720 are similar to steps 1610-1620 mentioned above. At step 1730, a third tracker attached to the subject measures the magnetic field emitted by the first tracker. At step 1740, the relative position of the first, second, and third trackers is determined based on the magnetic field measured by the second and third trackers. The changing field received by the trackers can be associated with the breathing states of the subject and used to generate a respiratory signal. At step 1750, the respiratory signal is generated based on the relative position of the first, second, and third trackers.

FIG. 18 is a flowchart of an exemplary method of generating a respiratory signal and a virtual representation of an intervention tool using an electromagnetic tracking system, such as those mentioned above. Steps 1810-1820 are similar to steps 1610-1620 mentioned above. At step 1830, a third tracker attached to the intervention tool measures the magnetic field emitted by the first tracker. At step 1840, the relative position of the first, second, and third trackers is determined based on the magnetic field measured by the second and third trackers. At step 1850, the respiratory signal is generated based on the relative position of the first and second trackers. At step 1860, the virtual representation of the intervention tool is generated based on the relative position of the first and third trackers.

FIG. 19 is a flowchart of an exemplary method of indicating when to advance an intervention tool during an intervention procedure on the subject (e.g., patient). At step 1910, a patient's respiration is monitored using an electromagnetic respiration monitor, such as those mentioned above. Next, while monitoring the patient's respiration, the patient is scanned using a scanner at step 1920 and an image data set is generated from the scan at step 1930. At step 1940, the image data set is associated with the respiratory state of the patient at the time of the scan. At step 1950, while continuing to monitor the patient's respiration using the electromagnetic respiration monitor, an intervention tool advancement indicator can indicate when to advance an intervention tool during a subsequent respiratory state of the patient that matches the respiratory state of the image data set. In this manner, the advancement indicator can indicate when to advance the intervention tool when the patient's target area is in the same position as when the scan was taken.

While the present invention has been illustrated by the description of embodiments thereof, and while the embodiments have been described in some detail, it is not the intention of the applicant to restrict or in any way limit the scope of the appended claims to such detail. Additional advantages and modifications will readily appear to those skilled in the art. The invention may take form in various compositions, components and arrangements, combinations and sub-combinations of the elements of the disclosed embodiments. Therefore, the invention in its broader aspects is not limited to the specific details, representative apparatus and methods, and illustrative examples shown and described. Accordingly, departures may be made from such details without departing from the spirit or scope of the applicant's general inventive concept.

1. An electromagnetic tracking system, comprising:
a first tracker attached to a subject, wherein the first tracker comprises an emitter to emit a magnetic field;
a second tracker attached to the subject, wherein the second tracker comprises a first receiver to measure the magnetic field; and
logic to determine a position of the second tracker relative to the first tracker based on the magnetic field measured by the first receiver and to generate a respiratory signal based on the position of the second tracker relative to the first tracker;
wherein the position of the second tracker relative to the first tracker changes during a plurality of respiratory states of the subject.

2. The system of claim 1, wherein the emitter comprises at least two emitting coils.

3. The system of claim 1, wherein the first receiver comprises at least two receiving coils.

4. The system of claim 1, wherein the first receiver comprises at least three receiving coils.

5. The system of claim 1, further comprising:
a third tracker attached to the subject, wherein the third tracker comprises a second receiver to measure the magnetic field; and
logic to determine a position of the third tracker relative to the first tracker based on the magnetic field measured by the second receiver and to generate the respiratory signal based on the position of the third tracker relative to the first tracker;
wherein the position of the third tracker relative to the first tracker changes during the plurality of respiratory states of the subject.

6. The system of claim 5, further comprising:
a fourth tracker, wherein the fourth tracker comprises a third receiver to measure the magnetic field; and
logic to determine a position of the fourth tracker relative to the first tracker based on the magnetic field measured by the third receiver and to generate the respiratory signal based on the position of the fourth tracker relative to the first tracker;
wherein the position of the fourth tracker relative to the first tracker changes during the plurality of respiratory states of the subject.

7. The system of claim 1, further comprising:
a fiducial marker, wherein the fiducial marker is viewable in an imaging data set from an imaging device to scan the subject, wherein the imaging data set is associated with a first respiratory state of the subject, and wherein the first respiratory state of the subject is one of the plurality of respiratory states of the subject.

8. The system of claim 7, further comprising:
a third tracker associated with an intervention tool, wherein the intervention tool is associated with an intervention procedure performed on the subject, and wherein the third tracker comprises a second receiver to measure the magnetic field; and
logic to determine a position of the third tracker relative to the fiducial marker based on the magnetic field measured by the second receiver.

9. The system of claim 7, further comprising:
a universal device, wherein the universal device comprises the fiducial marker and at least one of the first tracker and the second tracker.

10. An electromagnetic tracking system for indicating intervention tool advancement timing during an intervention procedure, comprising:
an electromagnetic tracking system for monitoring respiration of a subject, wherein the electromagnetic respiration monitor produces a respiratory signal indicative of a plurality of respiratory states of the subject; the electromagnetic respiration monitor comprising:
a first tracker attached to a subject, wherein the first tracker comprises an emitter to emit a magnetic field;
a second tracker attached to the subject, wherein the second tracker comprises a receiver to measure the magnetic field; and
logic to determine a position of the second tracker relative to the first tracker based on the magnetic field measured by the receiver and to generate the respiratory signal based on the position of the second tracker relative to the first tracker;
wherein the position of the second tracker relative to the first tracker changes during the plurality of respiratory states of the subject;
an imaging device for scanning the subject and generating an imaging data set, wherein the imaging data set is associated with a first respiratory state of the subject; and
an advancement indicator for indicating when to advance an intervention tool based on the respiratory signal, such that advancement of the intervention tool occurs during the first respiratory state of the subject.

11. The system of claim 10, further comprising:
a fiducial marker, wherein the fiducial marker is viewable in the imaging data set, and wherein the first respiratory state of the subject is one of the plurality of respiratory states of the subject.

12. The system of claim 11, further comprising:
a third tracker associated with the intervention tool, wherein the third tracker comprises a second receiver to measure the magnetic field; and
logic to determine a position of the third tracker relative to the fiducial marker based on the magnetic field measured by the second receiver.

13. The system of claim 11, further comprising:
a universal device, wherein the universal device comprises the fiducial marker and at least one of the first tracker and the second tracker.

14. A electromagnetic tracking method, comprising:
emitting a magnetic field from a first tracker, wherein the first tracker is attached to a subject and comprises an emitter to emit the magnetic field;
measuring the magnetic field with a second tracker, wherein the second tracker is attached to the subject and comprises a first receiver to measure the magnetic field;
determining a position of the second tracker relative to the first tracker based on the magnetic field measured by the first receiver; and
generating a respiratory signal based on the position of the second tracker relative to the first tracker;
wherein the position of the second tracker relative to the first tracker changes during a plurality of respiratory states of the subject.

15. The method of claim 14, further comprising:
measuring the magnetic field with a third tracker, wherein the third tracker is attached to the subject and comprises a second receiver to measure the magnetic field; and
determining a position of the third tracker relative to the first tracker based on the magnetic field measured by the second receiver; and
generating the respiratory signal based on the position of the third tracker relative to the first tracker;
wherein the position of the third tracker relative to the first tracker changes during a plurality of respiratory states of the subject.

16. The method of claim 14, further comprising:
generating an imaging data set from a scan of the subject with an imaging device, wherein the imaging data set is associated with a first respiratory state of the subject, and wherein the first respiratory state of the subject is one of the plurality of respiratory states of the subject.

17. The method of claim 16, further comprising:
providing a fiducial marker, wherein the fiducial marker is viewable in the imaging data set.

18. The method of claim 17, further comprising:
measuring the magnetic field with a third tracker, wherein the third tracker is associated with an intervention tool, wherein the intervention tool is associated with an intervention procedure performed on the subject, and wherein the third tracker comprises a second receiver to measure the magnetic field; and
determining a position of the third tracker relative to the fiducial marker based on the magnetic field measured by the second receiver.

19. The method of claim 18, further comprising:
generating a virtual representation of the intervention tool with an image of the subject, wherein the image is based on the imaging data set.

20. The method of claim 17, further comprising:
providing a universal marker, wherein the universal marker comprises the fiducial marker and at least one of the first tracker and the second tracker.
